(19) United States
(12) Patent Application Publication
(10) Pub. No.: US 2025/0099231 A1
(43) Pub. Date: Mar. 27, 2025

(54) PROSTHETIC HEART VALVE SYSTEMS AND METHODS WITH CORONARY OSTIUM ACCESS FEATURES

(71) Applicant: Medtronic, Inc., Minneapolis, MN (US)

(72) Inventors: Jeffrey D. Sandstrom, Scandia, MN (US); Ciaran McGuinness, Galway (IE); Karl L. Olney, Irvine, CA (US)

(21) Appl. No.: 18/729,837

(22) PCT Filed: May 19, 2023
(86) PCT No.: PCT/US2023/022811

§ 371 Date: Jul. 17, 2024

Related U.S. Application Data
(60) Provisional application No. 63/344,733, filed on May 23, 2022.

(51) Int. Cl. A61F 2/24

(52) U.S. Cl. CPC A61F 2/2409; A61F 2/2418

(57) ABSTRACT

Systems, devices and methods for maintaining coronary access after implanting a prosthetic heart valve and/or a transcatheter prosthetic heart valve replacement procedure. Systems of the present disclosure include a support structure and a valve structure. The support structure includes inner and outer frame members that define at least one auxiliary passageway. Upon final implantation, the valve structure is connected to the inner frame member, and the auxiliary passageway provides for passage of an auxiliary access device. In some embodiments, the inner frame member is formed or provided by a stent otherwise maintaining the valve structure. In other embodiments, the valve structure is secured to a stent of a prosthetic heart valve that is implanted separately from the support structure.

### FIELD

The present disclosure generally relates to prosthetic heart valves. More particularly, the present disclosure relates to prosthetic heart valve systems that facilitate access to nearby coronary arteries following implant.

### BACKGROUND

Following implantation of a prosthetic heart valve, a clinician may desire to access a region of the heart in close proximity to the prosthetic heart valve (e.g., native coronary arteries as part of a percutaneous coronary intervention (PCI) procedure). The implanted prosthetic heart valve may impede direct access to the native coronary artery or other region of interest. For example, the stent of a stented prosthetic heart valve (e.g., delivered via a transcatheter technique) may contact native anatomy (e.g., aortic root) in a manner presenting an obstruction to direct passage of an auxiliary access device to the region of interest. For example, rather than being directed around or outside of the stent, the auxiliary access device must instead be directed into the stent and then through an open cell. A similar concern can arise with some transcatheter techniques in which the native leaflets are not removed. Moreover, with some procedures in which a replacement prosthetic heart valve is deployed into a previously-implanted prosthetic heart valve, one or both of the structures of the previously-implanted heart valve and the replacement prosthetic heart valve can make it difficult to directly access nearby coronary arteries.

The present disclosure addresses problems and limitations associated with the related art and techniques.

### SUMMARY

The inventors of the present disclosure have observed that depending upon the particular patient anatomy (e.g., coronary height), prosthetic heart valve placement and other factors, displaced leaflets in a transcatheter aortic valve replacement procedure may become positioned against the aorta such that they block direct access to the coronary ostia and either acutely impede flow or provide challenging access to coronaries for future percutaneous coronary intervention procedures. In addition, or alternatively, the bulk of the native leaflets (or index valve leaflets in a prosthetic valve-in-prosthetic valve procedure) can reduce the Effective Orifice Area (EOA) of the prosthetic heart valve and, thus, can adversely affect gradients across the valve, particularly for patients having a relatively small valve annulus. For example, some patients have short and/or narrow coronary sinuses and the implantation of a prosthetic heart valve can block direct access to the coronary arteries, leading to serious complications. In instances where heart valve leaflets are trapped between two prosthetic heart valve devices, it can be more difficult to access the coronary artery ostia for future heart valve treatments. Aspects of this disclosure generally relate to spacer assemblies provided separately from or incorporated into prosthetic heart valves to define at least one channel outside of the prosthetic heart valve that provides a direct access path for an auxiliary access device, such as a guide catheter useful with a PCI procedure.

Some aspects of the present disclosure are directed to a system for treating a defective heart valve. The system includes a support structure and a valve structure. The support structure has a compressed configuration for delivery within a vasculature and an expanded configuration. The support structure includes an outer frame member, an inner frame member, and joint members. The outer frame member defines a central passage, a longitudinal axis, a first end, and a second end opposite the first end in a direction of the longitudinal axis. The joint members extend between and interconnect an interior face of the outer frame member and an exterior face of the inner frame member. The support structure is configured such that in the expanded configuration, at least a portion of the inner frame member is disposed within the central passage and is radially spaced from the interior face of the outer frame member by the joint members to establish a radial spacing. Further, at least one channel is defined along the radial spacing. The channel has a central angle of at least 30 degrees relative to the longitudinal axis in continuous extension between the first and second ends, and is free of the joint members. The system is configured such that upon final implantation, the outer frame member contacts native tissue and the valve structure is supported by the inner frame member. In some embodiments, the valve structure is provided as part of a prosthetic heart including a stent carrying the valve structure; with these and related embodiments, upon final implantation, the stent is secured to the inner frame member of the support structure. In other embodiments, the valve structure is permanently secured to the inner frame member such that the system serves as a prosthetic heart valve.

Other aspects of the present disclosure are directed to a method of treating a patient. The method includes implanting a system to a native heart valve of the patient. The system includes a support structure and a valve structure. The support structure has a compressed configuration for delivery within a vasculature and an expanded configuration. The support structure includes an outer frame member, an inner frame member, and joint members. The outer frame member defines a central passage, a longitudinal axis, a first end, and a second end opposite the first end in a direction of the longitudinal axis. The joint members extend between and interconnect an interior face of the outer frame member and an exterior face of the inner frame member. The support structure is configured such that in the expanded configuration, at least a portion of the inner frame member is disposed within the central passage and is radially spaced from the interior face of the outer frame member by the joint members to establish a radial spacing. Further, at least one channel is defined along the radial spacing. The channel has a central angle of at least 30 degrees relative to the longitudinal axis in continuous extension between the first and second ends, and is free of the joint members. Following the step of implanting, the outer frame member contacts native tissue and the valve structure is supported by the inner frame member. In some embodiments, methods of the present disclosure further include advancing an auxiliary access device, such as a catheter, through the channel, for example to access a native artery ostium.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a simplified side view of a system in accordance with principles of the present disclosure.

FIG. 2 is a top view of a support structure useful with the system of FIG. 1.

FIG. 3 is a cross-sectional view of the support structure of FIG. 2, taken along the line 3-3.

FIG. 4 is a top view of an alternate embodiment of a support structure useful with the system of FIG. 1.

FIG. 5 is a top view of another alternate embodiment of a support structure useful with the system of FIG. 1.

FIG. 6 is a cross-sectional view an alternate embodiment of a support structure similar to the support structure of FIG. 2, taken along the line 3-3.

FIGS. 7A and 7B illustrate deployment of the system of FIG. 1 to native anatomy to repair a defective heart valve.

FIG. 7C illustrates use of an auxiliary access device with the system of FIG. 1 following final implantation.

FIG. 7D illustrates a replacement prosthetic heart valve deployed to the implanted system of FIG. 1.

FIG. 8 is a simplified cross-sectional view of another system in accordance with principles of the present disclosure deployed to native anatomy to repair a defective heart valve.

FIG. 9 is a perspective view of another support structure in accordance with principles of the present disclosure and useful with the system of FIG. 1.

FIG. 10 illustrates deployment of the support structure of FIG. 9 to native anatomy in a region of an aortic heart valve.

FIG. 11 illustrates deployment of a prosthetic heart valve in the support structure of FIG. 10 deployed in native anatomy in a region of an aortic heart valve.

FIG. 12A is a simplified side view of a prosthetic heart valve useful with the system of FIG. 1.

FIG. 12B is an enlarged view of a portion of the prosthetic heart valve of FIG. 8A secured to a portion of the support structure of FIG. 1.

FIG. 13 is a perspective view of another support structure in accordance with principles of the present disclosure and useful with the system of FIG. 1.

FIG. 14 is a simplified cross-sectional view of another system in accordance with principles of the present disclosure upon final implantation, including a prosthetic heart valve connected to a support structure.

FIG. 15 is a simplified cross-sectional view of a delivery system for deployment of the system of FIG. 14.

FIG. 16 is a simplified cross-sectional view of another system in accordance with principles of the present disclosure upon final implantation, including a prosthetic heart valve connected to a support structure

FIG. 17 is a bottom view of the arrangement of FIG. 16 taken along line 17-17.

FIG. 18 is a simplified cross-sectional view of another system in accordance with principles of the present disclosure deployed to native anatomy to repair a defective aortic heart valve.

FIG. 19 illustrates delivery of the system of FIG. 18 to a target site.

FIG. 20 is a cross-sectional view of native anatomy in a region of an aortic heart valve.

FIG. 21 illustrates implantation of a sinus stent of the present disclosure to the anatomy of FIG. 20.

### DETAILED DESCRIPTION

Specific embodiments of the present disclosure are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician. As used herein, with reference to an stented prosthetic heart valve, the term “outflow” is understood to mean downstream to the direction of blood flow, and the term “inflow” is understood to mean upstream to the direction of blood flow.

Aspects of the disclosure are beneficial for use with prosthetic heart valves and heart valve repair methods including the implantation of a prosthetic heart valve, particularly, prosthetic heart valves delivered via a transcatheter procedure. As referred to herein, prosthetic heart valves can include a bioprosthetic heart valve structure having tissue leaflets or a synthetic heart valve having polymeric, metallic or tissue-engineered leaflets, and can be specifically configured for replacing or repairing valves of the human heart. In some embodiments, the valve of the human heart is an aortic valve, although the systems and methods of the present disclosure can be useful with the mitral, tricuspid, or pulmonary heart valve. The prosthetic heart valves of the present disclosure may be self-expandable, balloon expandable and/or mechanically expandable or combinations thereof. In general terms, the prosthetic heart valves of the present disclosure include a stent or stent frame having an internal lumen maintaining a valve structure (tissue or synthetic), with the stent frame having a normal, expanded condition or arrangement and collapsible to a compressed condition or arrangement for loading within the delivery device. For example, the stents or stent frames are support structures that include a number of struts or wire segments arranged relative to each other to provide a desired compressibility and strength to the prosthetic valve. The struts or wire segments are arranged such that they are capable of self-transitioning from, or being forced from, a compressed or collapsed arrangement to a normal, radially expanded arrangement. The struts or wire segments can optionally be formed from a shape memory material, such as a nickel titanium alloy (e.g., nitinol). The stent frame can be laser-cut from a single piece of material, or can be assembled from a number of discrete components.

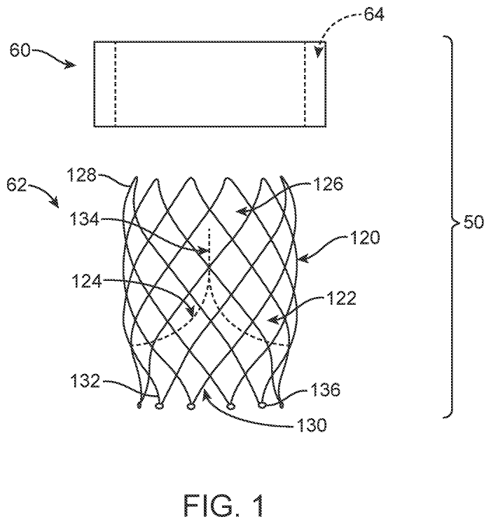

With the above in mind, one example of a system 50 in accordance with principles of the present disclosure is shown in simplified form in FIG. 1. The system 50 includes a support structure or spacer assembly 60 and a prosthetic heart valve 62. As described in greater detail below, in some embodiments, the support structure 60 is provided apart from the prosthetic heart valve 62 prior to implant. In other embodiments, the support structure 60 is integrally formed with the prosthetic heart valve 62. Regardless, upon final implantation, the support structure 60 and the prosthetic heart valve 62 are connected with one another, with the support structure 60 establishing and maintaining at least one auxiliary passageway or channel 64 (referenced generally) through which an auxiliary access device (not shown), such as a guide catheter, can pass. With this construction, upon final implant the support structure 60 is in contact with native tissue and the auxiliary passageway 64 provides access around or outside of the prosthetic heart valve 62 for advancement of the auxiliary access device, for example to access a coronary ostium proximate the implanted system 50 as described in greater detail below.

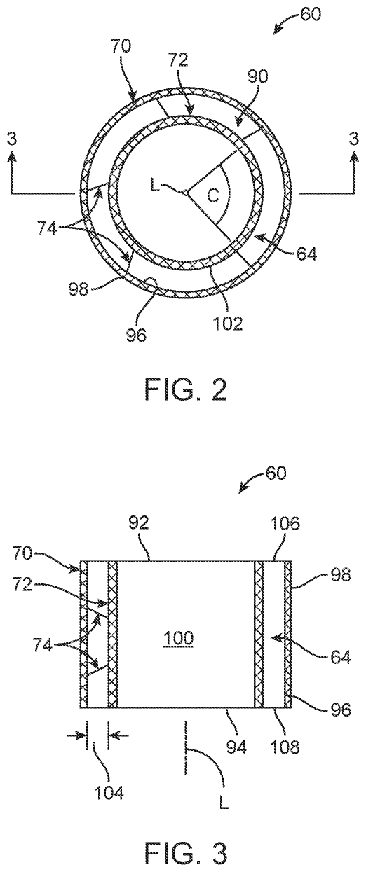

The support structure 60 can assume various forms. In general terms, and with reference to FIGS. 2 and 3, the support structure 60 can include a first or outer frame member 70, a second or inner frame member 72, and a plurality of joint members 74 (several of which are shown in FIGS. 2 and 3). As a point of reference, FIGS. 2 and 3 illustrate the support structure 60 in an expanded configuration. The support structure 60 is configured to be transitionable from the expanded configuration to a compressed configuration for delivery within the vasculature and vice-versa for implantation at a target site (e.g., the support structure 60 can have self-expanding construction, can be balloon expandable, mechanically expandable, etc.). Thus, in some embodiments, the outer frame member 70 and the inner frame member 72 can include a tubular stent or stent frame as is known in the art. Regardless, the outer frame member 70 defines a longitudinal axis L, a central passage 90 (referenced generally in FIG. 2), a first end (or outflow end) 92 and a second end (or inflow end) 94. The inflow end 94 is opposite the outflow end 92 relative to, or in a direction of, the longitudinal axis L. The central passage 90 is effectively defined by an interior face 96 of the outer frame member 70, with the interior face 96 being opposite an exterior face 98. While the outer frame member 70 is generally illustrated as having a shape of a right cylinder in the expanded configuration of FIGS. 2 and 3, other shapes (regular or irregular) are also acceptable. For example, a perimeter shape (and size) of the outer frame member 70 in the expanded state can be selected in accordance with an expected shape of the native anatomy to which the support structure 60 will be deployed as described below.

The inner frame member 72 is generally sized and shaped in accordance with a size and shape of the outer frame member 70, and defines a central passageway 100. The inner frame member 72 is constructed such that in the expanded configuration, at least a portion of the inner frame member 72 is sized and shaped to be disposed within the central passage 90 of the outer frame member 70, with that portion having a shape corresponding with that of a shape of the outer frame member 70 (e.g., the inner frame member 72 can have a right cylinder-like shape the same as or similar to the shape of the outer frame member 70 as reflected in FIG. 3). While FIG. 3 illustrates the outer frame member 70 and the inner frame member 72 having approximately the same height, and opposing ends of the inner frame member 72 being approximately aligned with the corresponding ends 92, 94 of the outer frame member 70, other constructions or relationships are also acceptable. For example, the inner frame member 72 can be sized and arranged to project (in a direction of the longitudinal axis L) outwardly beyond one or both of the ends 92, 94 of the outer frame member 70. In yet other embodiments, the inner frame member 72 can be provided by, or formed as a portion of, a stent component of the prosthetic heart valve 62 (FIG. 1) as described below.

The joint members 74 extend between and interconnect the interior face 96 of the outer frame member 70 and an exterior face 102 of the inner frame member 72. In the expanded configuration of FIGS. 2 and 3, the joint members 74 maintain the frame members 70, 72 relative to one another such that for at least that portion of the inner frame member 72 otherwise within the central passage 90 of the outer frame member 70, the inner frame member 72 is radially spaced from the interior face 96 of the outer frame member 70 by a radial spacing 104. The joint members 74 can assume various forms to enable transitioning between the compressed and expanded configurations, as well as for supporting the frame members 70, 72 relative to one another in the expanded configuration. The joint members 74 can be identical, or two (or more) differing formats can be employed. For example, one or more or all of the joint members 74 can be a strut (e.g., as conventionally used with stent constructions), wire segment, rivet, weld, etc.

Regardless of an exact configuration, the joint members 74 traverse the radial spacing 104 at various locations, with a sufficient number (and location) of the joint members 74 being provided to maintain the inner frame member 72 relative to the outer frame member 70 when subjected to expected forces following implant. An arrangement of the joint members 74 relative to the frame members 70, 72 is such that the at least one auxiliary passageway 64 is established along the radial spacing 104. The channel 64 extends between a first open side or outflow side 106 corresponding with the outflow end 92 of the outer frame member 70, and an opposing, second open side or inflow side 108 (that corresponds with the inflow end 94) and is free of, or does not include any of, the joint members 74 between the open sides 106, 108. Thus, the auxiliary passageway 64 is continuously open or unobstructed by any of the joint members 74 from the outflow side 106 to the opposing, inflow side 108. The spacing between the two, circumferentially-most proximate joint members 74 defines a circumferential extent of the auxiliary passageway 64, and can be identified as an arc relative to the annular or generally circular shape of the radial spacing 104 (in a plane perpendicular to the longitudinal axis L, such as that shown in FIG. 2). In some embodiments, the circumferential extent of the auxiliary passageway 64 defines a central angle C (relative to the longitudinal axis L) or arc angle of at least 12 degrees, alternatively at least 20 degrees, alternatively at least 30 degrees, alternatively at least 35 degrees, alternatively at least 40 degrees.

The support structure 60 can be configured to form one, two, or more of the auxiliary passageways 64 in the expanded state. Where two (or more) of the auxiliary passageways 64 are provided, a circumferential location of the auxiliary passageways 64 relative to one another can assume various forms. For example, the auxiliary passageways 64 can be equidistantly spaced, or can be more randomly located along the radial spacing 104. Regardless, the auxiliary passageway(s) 64 are sized to receive or allow passage of an auxiliary guide device, such as a PCI guide catheter. Thus, in some embodiments, the auxiliary passageway(s) 64 is optionally configured to receive, or allow passage of, a catheter with an internal diameter on the order of 6-8 Fr (e.g., a width of the radial spacing 104 can be in the range of 2-3 mm along with the minimum central angle C or arc angle as described above).

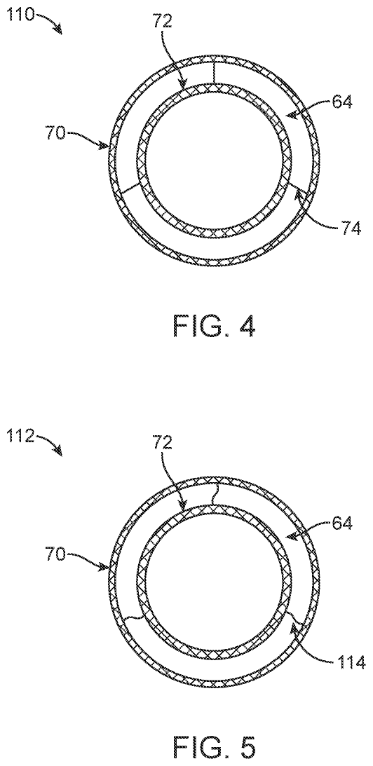

The joint members 74 and their arrangement relative to one another can assume a wide variety of other forms or formats that may or may not be directly implicated by the illustrations of FIGS. 2 and 3. For example, FIG. 4 illustrates, in simplified form, another support structure or spacer assembly 110 of the present disclosure that includes the outer and inner frame members 70, 72, and the joint members 74. In the view of FIG. 4, three of the joint members 74 are provided, approximately equidistantly spaced to define three of the auxiliary passageways 64 each having a central angle or arc on the order of 120 degrees. Additional joint members 74 can be provided, longitudinally aligned with the joint members 74 shown (and thus not visible or hidden in the view of FIG. 4). Another support structure or spacer assembly 112 of the present disclosure is shown, in simplified form, in FIG. 5. The support structure 112 includes the outer and inner frame members 70, 72 as described above, along with joint members 114. The joint members 114 can be similar to the joint members 74 (FIG. 2), but have an undulating shape in the expanded configuration. With these and similar configurations, the joint members 114 can more readily flex (e.g., to accommodate systole and diastole following implant, crimping during loading to a delivery device, etc.). Other joint member configurations that provide or promote flexibility and/or resilience are also acceptable, for example telescoping designs, expansion joint designs, etc.

Returning to FIG. 1, the prosthetic heart valve 62 can assume a variety of forms that may or may not be implicated by or shown in the illustration. As a point of reference, the prosthetic heart valve 62 is shown in a normal or expanded condition in the view of FIG. 1. The prosthetic valve 62 includes a stent or stent frame 120 and a valve structure 122 (referenced generally). The stent frame 120 can assume any of the forms mentioned above, and can be constructed to be self-expandable from a compressed condition for delivery through the vasculature to the normal, expanded condition. The stent 120 can be configured (e.g., sized and shaped) for implantation at any native valve of the human heart (e.g., mitral valve, aortic valve, tricuspid valve, or pulmonary valve).

The valve structure 122 of the prosthetic heart valve 62 can assume a variety of forms, and can be formed, for example, from one or more biocompatible synthetic materials, synthetic polymers, autograft tissue, homograft tissue, xenograft tissue, or one or more other suitable materials. In some embodiments, the valve structure 122 can be formed, for example, from bovine, porcine, equine, ovine, and/or other suitable animal tissues. In some embodiments, the valve structure 122 can be formed, for example, from heart valve tissue, pericardium, and/or other suitable tissue. In some embodiments, the valve structure 122 can include or form one or more leaflets 124. For example, the valve structure 122 can be in the form of a tri-leaflet bovine pericardium valve, a bi-leaflet valve, or another suitable valve.

In some prosthetic heart valve constructions, such as that of FIG. 1, the valve structure 122 can include two or three leaflets that are fastened together at enlarged lateral end regions to form commissural joints, with the unattached edges forming coaptation edges of the valve structure 122. The leaflets 124 can be fastened to a skirt that in turn is attached to the stent frame 120. The prosthetic heart valve 62 includes or defines an outflow portion 126 corresponding to a first or outflow end 128 of the prosthetic heart valve 62. The opposite end of the prosthetic heart valve 62 can define an inflow portion 130 corresponding to a second or inflow end 132 (receiving fluid) of the prosthetic heart valve 62. As shown, the stent frame 120 can have a lattice or cell-like structure, and optionally forms or provides posts corresponding with commissures 134 of the valve structure 122 as well as eyelets 136 (or other shapes; only a select few are labeled) at the outflow and inflow ends 128, 132.

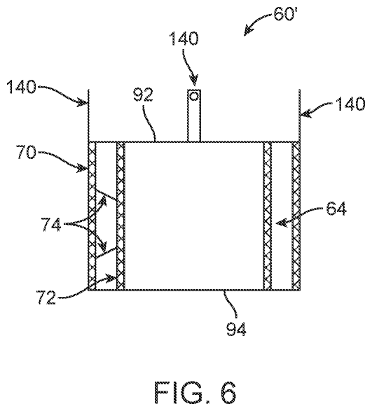

The system 50 can be used in various methods of treating a patient. With the non-limiting example of FIG. 1 in which the support structure 60 is physically separate from the prosthetic heart valve 62 prior to final implantation, some methods of the present disclosure include transitioning the support structure 60 to a compressed configuration and delivering the support structure 60 through a vasculature of the patient (e.g., known transcatheter techniques), and then deploying the support structure 60 to the expanded configuration at a target site proximate the heart valve to be repaired. In this regard, the spacer assemblies of the present disclosure can optionally include or incorporate one or more features that facilitate connection with a delivery device, for example in a manner that promotes recapture of the support structure when partially deployed from the delivery device. For example, FIG. 6 shows another support structure or spacer assembly 60′ of the present disclosure. In many respects, the support structure 60′ can be the same as or similar to the support structure 60 (FIGS. 1-3), and includes the frame members 70, 72 and the joint members 74. In addition, the support structure 60′ includes one or more paddles 140 extending from the first end 92 of the outer frame member 70. The paddle(s) 140 can assume various forms, and are generally configured for loading to a retainer component of a delivery device as is known in the art. As initially delivered to a target site, the support structure 60′ is radially compressed within the delivery device. During initial deployment, regions of the support structure 60′ proximate the second end 94 can be released from the delivery device and allowed to expand while the paddle(s) 140 remain secured to the delivery device. Under circumstances where the clinician desires to relocate the support structure 60′ relative to native anatomy prior to full deployment, because the paddles (140) remain connected to the delivery device, the support structure 60′ is readily recaptured by the delivery device (with the support structure 60′ being forced to a compressed condition). Once recaptured, the delivery device can be manipulated to relocate the support structure 60′ relative to native anatomy and then effect full deployment.

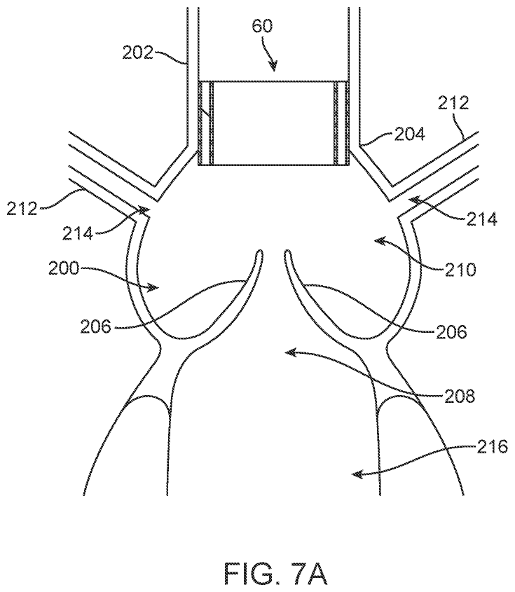

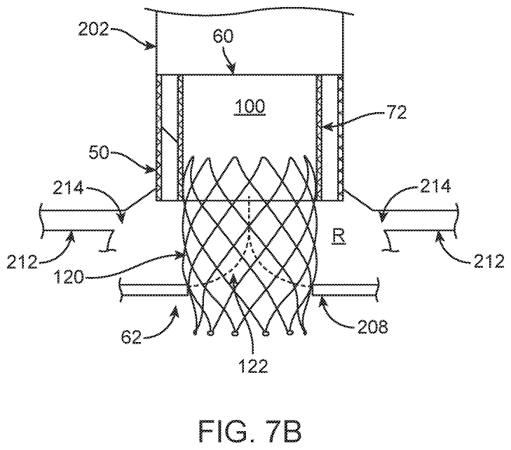

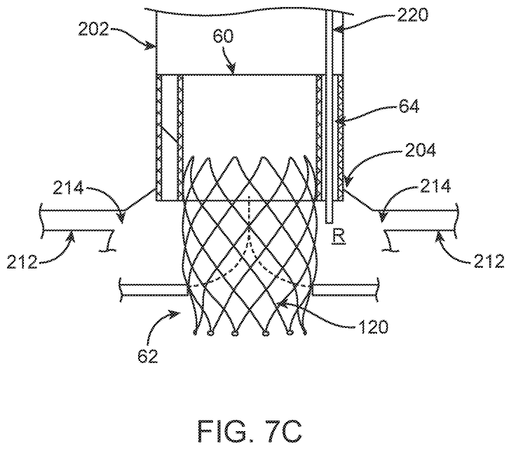

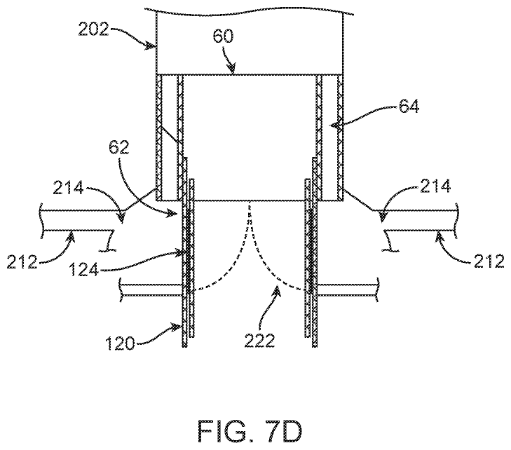

Regardless of whether recapture or other delivery/deployment features are provided, the systems of the present disclosure, for example the system 50 of FIG. 1, can be deployed at a region of an aortic valve in a manner that promotes access to coronary arteries. For example, FIG. 7A illustrates the support structure 60 implanted to native anatomy proximate an aortic heart valve 200. Generally, patient anatomy at and adjacent the aortic valve 200 includes an aorta 202, sinotubular junction (“STJ”) 204, native valve leaflets 206, aortic valve annulus 208, sinus region 210, coronary arteries (or “coronaries”) 212 each having a coronary ostium 214, and left ventricle 216. As shown, the support structure 60 can be deployed along the aorta 202 in a region of the STJ 204 such that support structure 60 engages native tissue and is held firmly in place. The outer frame member 70 is in direct contact with and engages native tissue of the aorta 202 and/or the STJ 204. The prosthetic heart valve 62 is similarly transitioned to the compressed condition and delivered through the vasculature to the aortic heart valve 200 and deployed as generally reflected by the simplified representation of FIG. 7B. For pictorial simplicity, the leaflets 206 (FIG. 7A) are omitted from the view of FIG. 7B, and anatomical structures of the aortic valve annulus 208 and the coronary arteries 212 are drawn generally. Upon final implantation of the system 50, the stent 120 of the prosthetic heart valve 62 contacts or engages the inner frame member 72 of the support structure 60. Thus, the valve structure 122 of the prosthetic heart valve 62 is supported by the inner frame member 72. Blood flow readily occurs through the support structure 60 to/from the prosthetic heart valve 62 via at least the lumen 100.

At a later point in time, a clinician may desire to access, via the aorta 202, a region anatomically between the prosthetic heart valve 62 and native tissue. One such region is generally indicated at “R” in FIG. 7B. For example, the region R may be proximate one of the coronary artery ostia 214, and by which a targeted one of the coronary artery ostium 214 can be accessed. Under these circumstances, and with reference to FIG. 7C, an auxiliary access device 220 (e.g., a guide catheter) can be delivered through the aorta 202 and readily advanced through the channel 64 to or toward the region R, unimpeded by the stent 120, gaining access to the coronary ostium 214. In other words, were the support structure 60 not present and the stent 120 expanded into contact with aorta 202, STJ 204, or other native tissue, in order to access the coronary ostium 214, the auxiliary access device 220 would likely need to be directed into the stent 120, and then outwardly therefrom via an open cell of the stent 120. By establishing and maintaining the auxiliary passageway(s) 64 following implant, the support structure 60 facilitates direct access to one or more regions proximate the prosthetic heart valve 62. With embodiments in which the support structure 60 forms two or more of the auxiliary passageways 64, access to a number of different regions, for example all of the coronary ostia 214, is afforded and/or it is likely that at least one of the auxiliary passageways 64 will be generally located near a particular region of interest regardless of how the support structure 60 happens to be rotationally oriented relative to the native tissue during the delivery and deployment steps.

The systems of the present disclosure, such as the system 50 (FIG. 1), can provide benefits for patients who subsequently require a replacement valve. FIG. 7D generally illustrates an optional, subsequent procedure in which a replacement prosthetic heart valve 222 has been deployed into the previously-implanted prosthetic heart valve 62 (represented in simplified form in FIG. 7D). Under these circumstances, the support structure 60 remains in place, with the auxiliary passageway(s) 64 continuing to provide direct access through the aorta 202 to the coronary arteries 212 or other regions outside of the prosthetic heart valve 62 (e.g., an auxiliary access device can be advanced through the channel 64 to access the ostium 214 of the targeted coronary artery 212). Thus, even if the replacement prosthetic heart valve 222 traps the leaflets 124 of the previously-implanted prosthetic heart valve 62 against the stent 120 in a manner that might otherwise obstruct open cells of the stent 120, a clinician can continue to easily access the targeted coronary artery 212 via the auxiliary passageway 64.

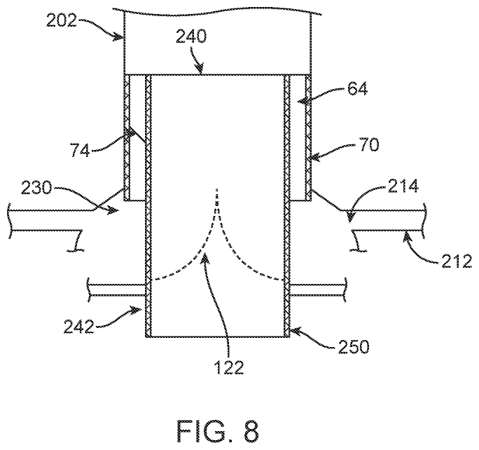

While the system 50 provides the support structure 60 and the prosthetic heart valve 62 as separate components prior to implantation, in other embodiments the support structure 60 can be integrally formed with the prosthetic heart valve 62. For example, FIG. 8 is a simplified representation of another embodiment system 230 in accordance with principles of the present disclosure and implanted to repair the heart valve 200. The system 230 can be the same as or similar to the system 50 (FIG. 1) described above, and generally includes a support structure or spacer assembly 240 integrally formed with, or carried by, a prosthetic heart valve 242. The support structure 240 and the prosthetic heart valve 242 can be similar to the descriptions above, with the support structure 240 including the outer frame member 70. The prosthetic heart valve 242 includes a stent 250 to which the valve structure 122 is secured. The stent 250 is generally similar to the stent 120 (FIG. 1) described above. With the system 230, however, the stent 250 has an elongated shape, and is directly connected to the outer frame member 70 by the joint members 74 as described above, with the joint members 74 arranged to establish one or more of the auxiliary passageways 64. Thus, a portion of the stent 250 effectively serves as the inner frame member 72 (FIG. 2). During use, the system 230 is collectively transitioned to a compressed configuration for delivery through the vasculature, and deployed to the final implantation arrangement as shown. As with previous embodiments, by establishing and maintaining the auxiliary passageway(s) 64 following implant, the support structure 240 facilitates direct access from the aorta 202 to one or more regions proximate the heart valve 242 (e.g., the ostium 214 of a targeted coronary artery 212) as described above.

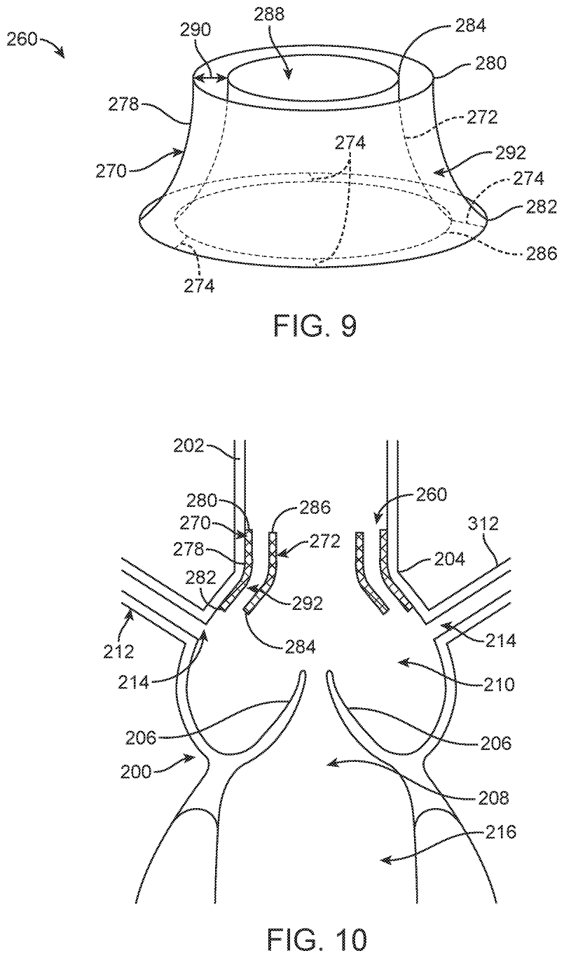

Another embodiment of a support structure or spacer assembly 260 useful with the systems of the present disclosure, such as the system 50 (FIG. 1), is shown in FIG. 9. The support structure 260 includes a first or outer frame member 270, a second or inner frame member 272, and a plurality of joint members 274 (several of which are shown in FIG. 9). As a point of reference, FIG. 7 illustrates the support structure 260 in an expanded configuration. The support structure 260 is configured to be transitionable from the expanded configuration to a compressed configuration for delivery within the vasculature and vice-versa for deployment and implantation at a target site (e.g., the support structure 260 can have self-expanding construction, can be balloon expandable, mechanically expandable, etc.).

The outer frame member 270 can be the same as or similar to the outer frame member 70 (FIG. 1) as described above, and thus can be a tubular stent or stent frame as is known in the art. The outer frame member 270 defines an exterior face 278, and a first end 280 opposite a second end 282, and in the example shown, is configured to (in the expanded configuration) define a conical-like shape, with an expanding diameter more prominent in a region of the second end 282. A diameter of the second end 282 is greater than a diameter of the first end 280. With these and similar constructions, the outer frame member 270 can be sized and shaped for implant at one more native anatomies of interest, for example along the aorta in a region of the sinotubular junction.

The inner frame member 272 can be the same as or similar to the inner frame member 72 (FIG. 1) as described above, and thus can be a tubular stent or stent frame as known in the art. The inner frame member 272 is generally sized and shaped in accordance with a size and shape of the outer frame member 270. More particularly, the inner frame member 272 is constructed such that in the expanded configuration, at least a portion of the inner frame member 272 is sized and shaped to be disposed within the outer frame member 270, with that portion having a shape corresponding with the shape of the outer frame member 270 (e.g., the inner frame member 272 can have the conical-like shape that is the same as or similar to the shape of the outer frame member 270). While FIG. 9 illustrates the outer frame member 270 and the inner frame member 272 having approximately the same height, and opposing ends 284, 286 of the inner frame member 272 being approximately aligned with the corresponding ends 280, 282 of the outer frame member 270, other constructions or relationships are also acceptable as described above. Regardless, the inner frame member 272 defines a lumen 288 in at least the expanded configuration.

The joint members 274 can be the same as or similar to the joint members 74 (FIG. 2) as described above, and extend between and interconnect an interior face of the outer frame member 270 and an exterior face of the inner frame member 272. In the expanded configuration of FIG. 9, the joint members 274 maintain the frame members 270, 272 relative to one another such that for at least that portion of the inner frame member 272 otherwise within the outer frame member 270, the inner frame member 272 is radially spaced from the interior face of the outer frame member 270 by a radial spacing 290. The joint members 274 can assume various forms to enable transitioning between the compressed and expanded configurations, as well as for supporting the frame members 270, 272 relative to one another in the expanded configuration. The joint members 274 can be identical, or two (or more) differing formats can be employed. For example, one or more or all of the joint members 274 can be a strut (e.g., as conventionally used with stent constructions), wire segment, rivet, weld, etc.

Regardless of an exact configuration, the joint members 274 traverse the radial spacing 290 at various locations, with a predetermined number (and location) of the joint members 274 being provided to maintain the inner frame member 272 relative to the outer frame member 270 when subjected to expected forces following implant. An arrangement of the joint members 274 relative to the frame members 270, 272 is such that the at least one auxiliary passageway 292 (referenced generally) is established along the radial spacing 290. Commensurate with the descriptions above, the auxiliary passageway(s) 292 extends between opposing open sides corresponding with the ends 280, 282 of the outer frame member 270, and is free of, or does not include any of, the joint members 274 between the open sides. A circumferential extent of the auxiliary passageway 292 defines a central angle C or arc angle (not labeled in FIG. 9, but identified, for example, in FIG. 2) of at least 12 degrees, alternatively at least 20 degrees, alternatively at least 30 degrees, alternatively at least 35 degrees, alternatively at least 40 degrees.

The support structure 260 can be configured to form one, two, or more of the auxiliary passageways 292 in the expanded state. Where two (or more) of the auxiliary passageways 292 are provided, a circumferential location of the auxiliary passageways 292 relative to one another can assume various forms. For example, the auxiliary passageways 292 can be equidistantly spaced, or can be more randomly located along the radial spacing 290. Regardless, the auxiliary passageway(s) 292 are sized to receive or allow passage of an auxiliary guide device, such as a PCI guide catheter. Thus, in some embodiments, the auxiliary passageway(s) 292 is optionally configured to receive, or allow passage of, a catheter with an inner diameter on the order of 6-8 Fr (e.g., a width of the radial spacing 290 can be in the range of 2-3 mm).

In some embodiments, the support structure 260 is useful with systems for treating a native aortic heart valve. In this regard, FIG. 10 illustrates one example of the support structure 260 implanted to native anatomy proximate the aortic heart valve 300. As shown, the support structure 260 can be deployed along the aorta 202 in a region of the STJ 204, with a shape of the outer frame member 270 generally mimicking the corresponding anatomical shape (e.g., at least a region of the outer frame member 270 has a diameter in the expanded configuration that is larger than the expected size or diameter of the STJ 204 such that support structure 260 engages native tissue and is held firmly in place).

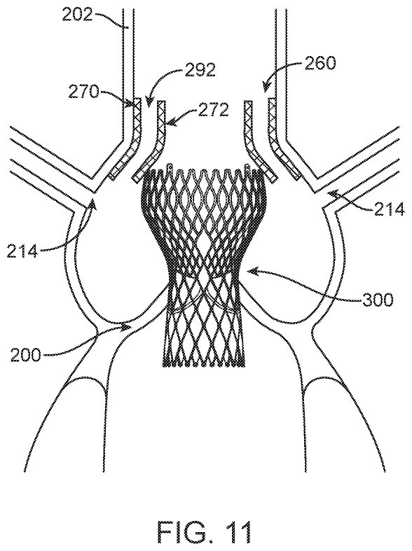

With additional reference to FIG. 11, a prosthetic heart valve 300 (e.g., the same as or similar to the prosthetic heart valve 62 (FIG. 1) described above) can then be deployed to the aortic heart valve 200. Commensurate with the descriptions above, a stent of the so-deployed prosthetic heart valve 300 contacts or engages the inner frame member 272 such that the inner frame member 272 supports the valve structure of the prosthetic heart valve 300. Following final implantation of the system, the auxiliary passageway(s) 292 facilitate direct access to the coronary ostia 214 via a vascular approach through the aorta 202, with the auxiliary access device (e.g., guide catheter) being directed from the aorta 202, through one of the auxiliary passageways 292, and to the targeted coronary ostium 214.

In some examples, the support structure 260 is well-suited for systems including a tall prosthetic aortic heart valve, better ensuring that the outflow of the prosthetic heart valve would not block off access to the coronaries 212. “Tall” prosthetic heart valves can be considered, for example, as those having a height that contacts the sinotubular junction 204 in the expanded condition when deployed at the aortic valve 200. Further, presence of the support structure 260 can lower the forces of prosthetic heart valve deployment by reducing a distance the outflow end of the prosthetic heart valve needs to travel before contacting the anatomy. The support structure 260 can further act as a visualization aid under fluoroscopy for physicians to know where to deploy the prosthetic heart valve, thus improving conduction disturbances and paravalvular leakage (“PVL”) rates.

Returning to FIGS. 1-3, in some embodiments the support structure 60 and the prosthetic heart valve 62 can include or incorporate complementary mating features that more robustly connect or dock the prosthetic heart valve 62 to the support structure 60 upon final implantation of the system 50. In some embodiments, a protrusion (e.g., hook, barb, post, bar, rib, etc.) can be provided with one of the support structure 60 or the prosthetic heart valve 62 that is configured to be captured within or by an aperture provided with the other of the support structure 60 or the prosthetic heart valve 62. For example, with embodiments in which the inner frame member 72 is a stent or stent-like body, the inner frame member 72 will have a lattice structure with a number of open cells or apertures. The corresponding prosthetic heart valve 62 can, in turn, be provided with or carry one or more protrusions sized and shaped to be captured within a corresponding one of the open cells.

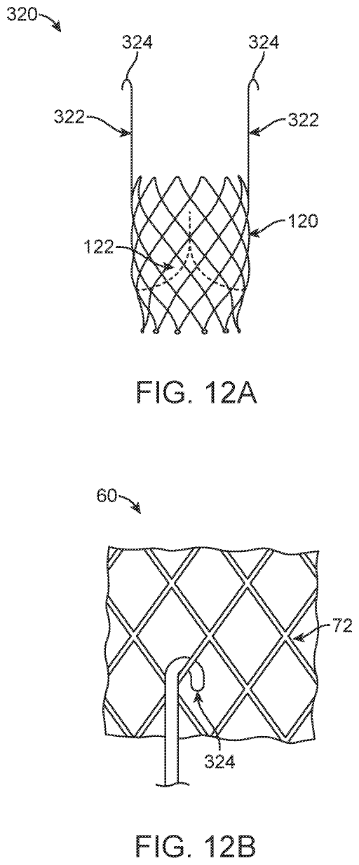

In FIG. 12A, for example, a prosthetic heart valve 320 useful with the systems of the present disclosure, for example with the support structure 60 (FIGS. 2 and 3) is shown in simplified form. In this example, the prosthetic heart valve 320 can be the same as or similar to the prosthetic heart valve 62 (FIG. 1) described above, including the stent 120 maintaining the valve structure 122 (referenced generally). The prosthetic heart valve 320 further includes one or more protrusions 322. The protrusions 322 can be formed by, or attached to, the stent 120, and include an arm extending away from the valve structure 122 and terminating at an end forming a hook 324. Other engagement type structures can be employed in place of the hook 324, such as a bar or snap-fit feature. Regardless, following deployment of the support structure 60 (e.g., the arrangement of FIG. 7A), the prosthetic heart valve 320 can be delivered in a compressed condition (including the protrusions collapsed within the delivery device) to the target site and deployed. During the deployment procedure, the prosthetic heart valve 320 can be manipulated to bring the hooks 324 into engagement with corresponding ones of the apertures or open cells in the inner frame member 72 as generally illustrated in FIG. 12B to more robustly connect the prosthetic heart valve 320 with the support structure 60.

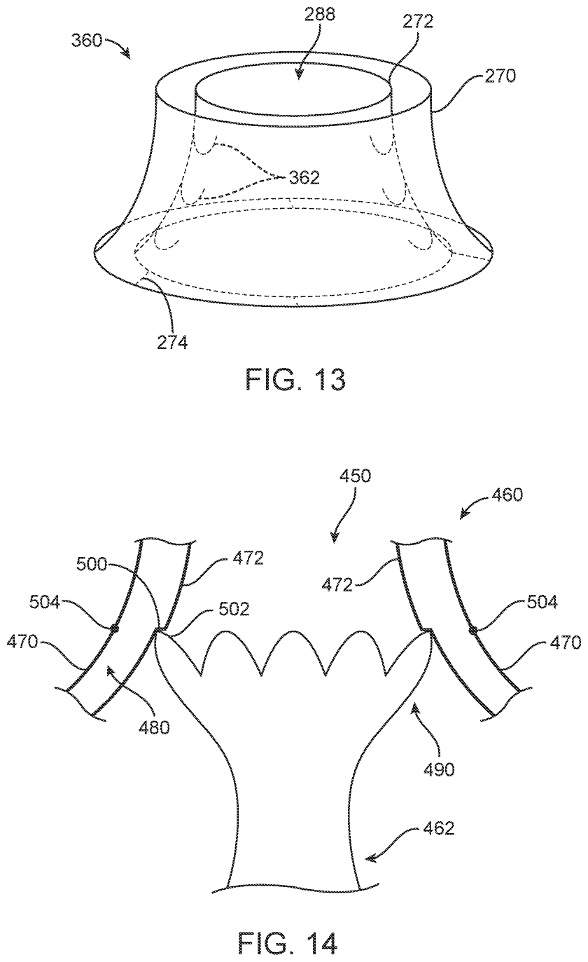

Alternatively or in addition, protrusions can be provided with the support structure 60. FIG. 13 depicts another example support structure or spacer assembly 360 useful with the systems of the present disclosure, for example with the prosthetic heart valve 62 (FIG. 1). The support structure 360 can optionally be the same as or similar to the support structure 260 (FIG. 9) described above, and includes the outer frame member 270, the inner frame member 272 and the joint members 274 as previously described. In addition, one or more protrusions 362 are formed or carried by the inner frame member 272. Each of the protrusions 362 are generally configured to engage with, or be captured within, a corresponding aperture provided with the prosthetic heart valve 62, for example cell openings formed by the stent 120 (FIG. 1). For example, one or more or all of the protrusions 362 can be a hook or hook-like structure, projecting generally inwardly into the lumen 288 formed by the inner frame member 272. Following deployment of the support structure 360 (e.g., the arrangement of FIG. 7A), the prosthetic heart valve 62 can be delivered in a compressed condition to the target site and deployed. During the deployment procedure, the prosthetic heart valve 62 can be manipulated to bring one or more of the hooks 362 into engagement with a corresponding ones of the apertures or open cells in the stent 120 to more robustly connect the prosthetic heart valve 62 with the support structure 360.

Portions of another embodiment system 450 of the present disclosure are shown in simplified form in FIG. 14, and illustrate another example of optional complementary engagement features of the present disclosure. The system 450 includes a support structure or spacer assembly 460 and a prosthetic heart valve 462. The support structure 460 can generally assume any of the forms described elsewhere, configured to be transitionable from the expanded configuration of FIG. 14 to a compressed configuration and vice-versa. The support structure 460 includes a first or outer frame member 470, a second or inner frame member 472, and joint members (not shown) that can assume any of the forms described above, for example the outer frame member 70, the inner frame member 72, and the joint members 74 of FIGS. 2 and 3. Commensurate with the descriptions above, in the expanded configuration of FIG. 14, the support structure 460 establishes one or more auxiliary passageways 480. The prosthetic heart valve 462 can assume any of the forms described above, and generally includes at least a stent 490 (illustrated in simplified form) maintaining a valve structure (not shown).

With the embodiment of FIG. 14, the inner frame member 472 forms or defines, in the expanded configuration, a ledge or shoulder 500 configured to receive an edge 502 of the prosthetic heart valve 462, for example an outflow end of the stent 490, upon final implantation. The ledge 500 can be annular, extending between or defining an inner diameter and an outer diameter. A geometry of the ledge 500 can be selected in accordance with an expected diameter of the edge 502 in the expanded condition of the prosthetic heart valve 462. For example, the inner diameter of the ledge 500 is selected to be less than the expected diameter of the edge 502 (again, in the expanded condition), whereas the outer diameter of the ledge 500 can approximate or be greater than the expected diameter of the edge 502. With this construction, following deployment of the support structure 460 to a target site (e.g., the arrangement of FIG. 7A), the prosthetic heart valve 462 can be transitioned to the expanded condition and directed into engagement with the support structure 460.

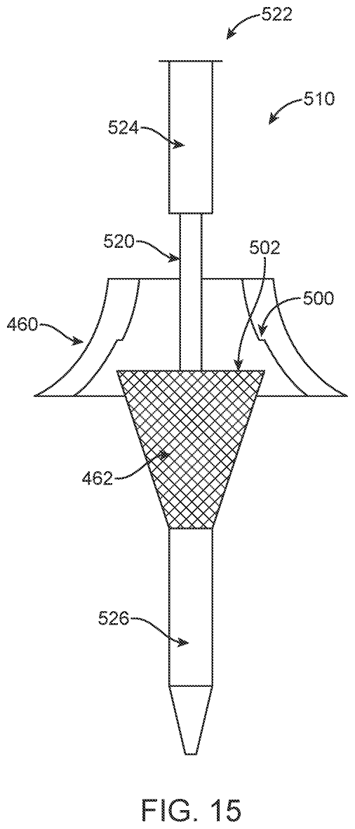

For example, FIG. 15 provides a simplified illustration of a delivery device 510 useful for delivering and deploying the prosthetic heart valve 462 relative to an anatomical target site (e.g., a native aortic valve) and the support structure 460. The delivery device 510 can be the same as or similar to conventional transcatheter valve delivery devices, and generally includes an inner shaft assembly 520 and an outer shaft assembly 522 (referenced generally). The inner shaft assembly 520 is co-axially received within the outer shaft assembly 522, and incorporates one or more features (hidden) for connection to the prosthetic heart valve 462. The outer shaft assembly 522 generally includes or forms an outer sheath or capsule defined by first and second capsule segments 524, 526. In an initial delivery state, prosthetic heart valve 462 is compressed or crimped over the inner shaft assembly 520, and the outer shaft assembly 522 is arranged such that the first and second capsule segments 524, 526 abut one another and contain an entirety of the prosthetic heart valve 462. A distal region of the prosthetic heart valve 462 is connected to the inner shaft assembly 520. The delivery device 510 is manipulated to direct the compressed and contained prosthetic heart valve 462 through the patient's vasculature to a target site at a region of the aortic valve. In this regard, the compressed and contained prosthetic heart valve 462 is located distally beyond the previously-implanted support structure 460. The outer shaft assembly 522 is then operated to proximally retract the first capsule segment 524 relative to the inner shaft assembly 520 and the second capsule segment 526 to the arrangement of FIG. 15. With operation, a section of the prosthetic heart valve 462 is released from the confines of the delivery device 510 and is allowed to self-expand as reflected by FIG. 15. A remainder of the prosthetic heart valve 462 is within the second capsule segment 526 and remains connected to the inner shaft assembly 520. From the arrangement of FIG. 13, then, the delivery device 510 can be manipulated to move the prosthetic heart valve 462 toward the support structure 460 (i.e., upwardly relative to the orientation of FIG. 15), bringing the edge 502 into contact with the ledge 500 to attain the arrangement of FIG. 14. Interface between the edge 502 and the ledge 500 can provide a more robust connection between the support structure 460 and the prosthetic heart valve 462. The delivery device 510 can then be operated to distally advance the second capsule segment 526 relative to the prosthetic heart valve 462, allowing the prosthetic heart valve 462 to fully deploy. Other delivery device configurations capable of releasing a proximal section of the prosthetic heart valve 462 (i.e., the section proximate or facing the previously-implanted support structure 460) are also acceptable; for example, suture-based delivery device.

Optionally, and with reference to FIG. 14, additional engagement features, such as the protrusions (e.g., hooks) described above, can be provided. Regardless, an interface between the ledge 500 and the edge 502 serves to minimize possible migration of the prosthetic heart valve 462 upwardly relative to the support structure 460 (relative to the orientation of FIG. 14). Thus, for example, where the support structure 460 is implanted within the aorta proximate the STJ and the prosthetic heart valve 462 is implanted to repair the aortic heart valve, the arrangement of FIG. 14 can minimize or prevent migration of the prosthetic heart valve 462 into the aorta. In some embodiments, the support structure 460 can optionally include one or more markers 504 (e.g., radiopaque material) proximate the ledge 500 (e.g., formed by or attached to the outer frame member 470. Where provided, the marker(s) 504 can assist a clinician in identifying a desired depth, canting and/or rotational alignment of the prosthetic heart valve 462 during the implantation procedure.

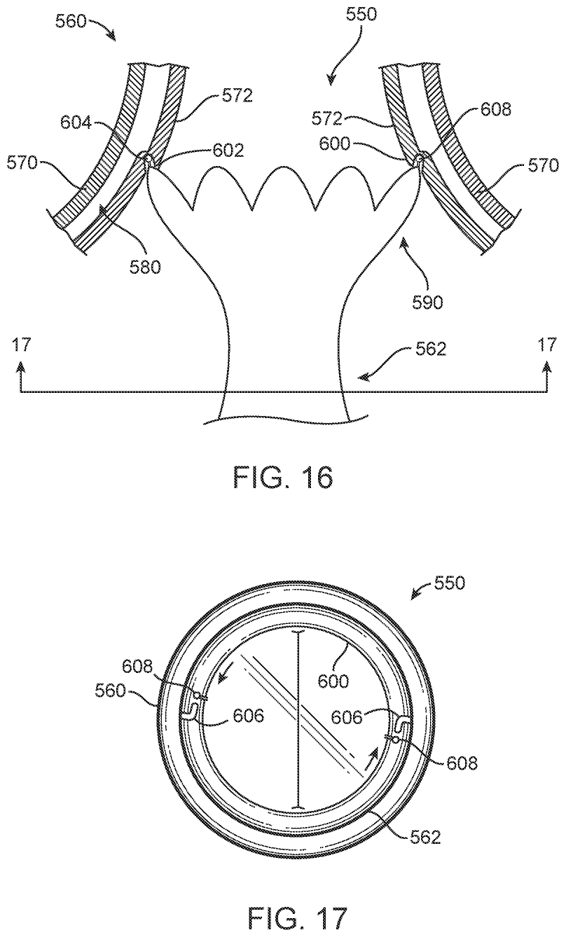

Portions of another embodiment system 550 are shown in simplified form in FIG. 16, and illustrate another example of optional complementary engagement features of the present disclosure. The system 550 includes a support structure or spacer assembly 560 and a prosthetic heart valve 562. The support structure 560 can generally assume any of the forms described elsewhere, configured to be transitionable from the expanded configuration of FIG. 16 to a compressed configuration and vice-versa. The support structure 560 includes a first or outer frame member 570, a second or inner frame member 572, and joint members (not shown) that can assume any of the forms described above, for example the outer frame member 70, the inner frame member 72, and the joint members 74 of FIGS. 2 and 3. Commensurate with the descriptions above, in the expanded configuration of FIG. 16, the support structure 560 establishes one or more auxiliary passageways 580. The prosthetic heart valve 562 can assume any of the forms described above, and generally includes at least a stent 590 (illustrated in simplified form) maintaining a valve structure (not shown).

With the embodiment of FIG. 16, the inner frame member 572 forms or defines, in the expanded configuration, a ledge or shoulder 600 configured to receive an edge 602 of the prosthetic heart valve 562, commensurate with a relationship between the ledge 500 (FIG. 14) and the edge 502 (FIG. 14) as described above. In addition, the inner frame member 572 defines a slot 604 that is open to the ledge 600. As best seen in FIG. 17, one or more hooks 606 are carried by the inner frame member 572 within the slot 604. The prosthetic heart valve 562 includes one or more complimentary features configured to selectively interface with the hooks 606 when disposed within the slot 604. For example, one or more paddles or similar structures 608 can extend from the edge 602. The number of, and circumferential spacing between, the paddles 608 corresponds with the number of, and circumferential spacing between, the hooks 606. With this construction, following deployment of the support structure 560 to a target site (e.g., the arrangement of FIG. 7A), the prosthetic heart valve 562 can be transitioned to the expanded condition and directed into engagement with the support structure 560 (e.g., using the delivery device 510 and techniques described above with respect to FIG. 15). As the prosthetic heart valve 562 is moved toward the support structure 560 (i.e., upwardly relative to the orientation of FIG. 16), the paddles 608 enter the slot 604 and the edge 602 comes into contact with the ledge 600. From this arrangement, the prosthetic heart valve 562 is rotated relative to the support structure 560 (i.e., counterclockwise relative to the orientation of FIG. 17), until the paddles 608 are captured within a corresponding one of the hooks 606. Further, the paddles 608 enter the slot 604 thus providing a more robust connection between the support structure 560 and the prosthetic heart valve 562. As a point of reference, with embodiments in which a delivery device that is the same as or similar to the delivery device 510 of FIG. 15 is used to deliver and deploy the prosthetic heart valve 562, the second capsule segment 526 can remain over the prosthetic heart valve 562 as the delivery device 510 is manipulated to bring the paddles 608 into the slot 604 and then rotate the prosthetic heart valve 562 to secure the paddles 608 with respective ones of the hooks 606. Once the paddles 608 are secured to the hooks 606, the second capsule segment 526 can be displaced from over the prosthetic heart valve 562 (thus permitting full deployment) by distally advancing the second capsule segment 526.

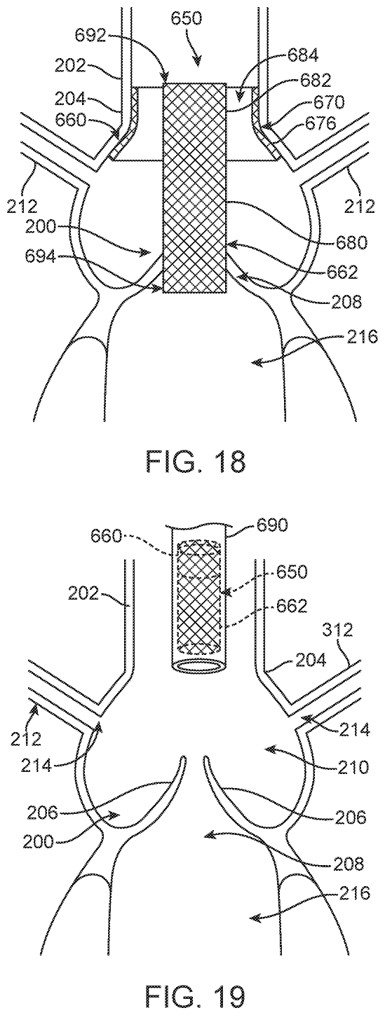

Another system 650 in accordance with principles of the present disclosure is shown in FIG. 18 optionally implanted to the anatomy of the aortic heart valve 200. The system 650 can be the same as or similar to the system 230 (FIG. 8), and includes a support structure or spacer assembly 660 integrally formed with, or carried by, a prosthetic heart valve 662. The support structure 660 and the prosthetic heart valve 662 can be the same as or similar to the descriptions above, with the support structure 660 including a first or outer frame member 670 defining an exterior face 676. The prosthetic heart valve 662 includes a stent 680 to which a valve structure (not shown) is secured. A region 682 of the stent 680 extends into the outer frame member 670, and can be designated as a second or inner frame member. The inner frame member 682 of the stent 680 is directly connected to the outer frame member 670 by joint members (not shown, but the same as or similar to the joint members 74 (FIGS. 2 and 3) described above), with the joint members arranged to establish one or more auxiliary passageways 684 in the expanded configuration commensurate with other embodiments of the present disclosure.

In some embodiments, the system 650 can be configured for repairing an aortic valve as reflected by FIG. 18. For example, the outer frame member 670 can be configured (e.g., sized and shaped) to be deployed or implanted along the aorta 202 in a region of the STJ 204, with the exterior face 676 contacting native tissue. Further, a length of extension of the stent 680 from the outer frame member 670, as well as a location of the valve structure (not shown) along the stent 680, can be selected to locate the valve structure (not shown) at the aortic valve annulus 208 with the outer frame member 670 secured at the STJ 304. In the implantation arrangement of FIG. 18, the system 650 serves to repair or replace the aortic valve 200, with the auxiliary passageway(s) 684 facilitating access to, for example, the coronary arteries 212 via the aorta 202 commensurate with the descriptions above. The system 650 can alternatively incorporate other features and/or geometry appropriate for implantation to a mitral valve, tricuspid valve, or pulmonary valve.

As explained above, the outer frame member 670 is permanently secured to the stent 680 prior to delivery placement within the patient. With this construction, an entirety of the system 650 can be compressed within a catheter 690 or similar device for delivery through the vasculature to a target site as shown in FIG. 19. The support structure 660 and the prosthetic heart valve 662 can be delivered and deployed in a single procedure and process, thus reducing procedure time and increasing ease of the procedure. Following final implantation (i.e., the arrangement of FIG. 18), increased radial force applied to an outflow end 692 of the stent 680 (via attachment to the outer frame member 670 that in turn is held against the aorta 202) can assist in limiting possible migration of the prosthetic heart valve 662 after deployment. Moreover, because at least some fixation-type support is provided to the outflow end 692, the stent 680 can be configured to exhibit a lesser radial force at an inflow end 694 (as compared to conventional transcatheter prosthetic heart valves). This attribute could, in turn, mean a reduction in conduction disturbances is possible. A replacement prosthetic heart valve could later be placed within the stent 680, if needed, while maintaining access to the coronary ostia 314 via the auxiliary passageway(s) 684.

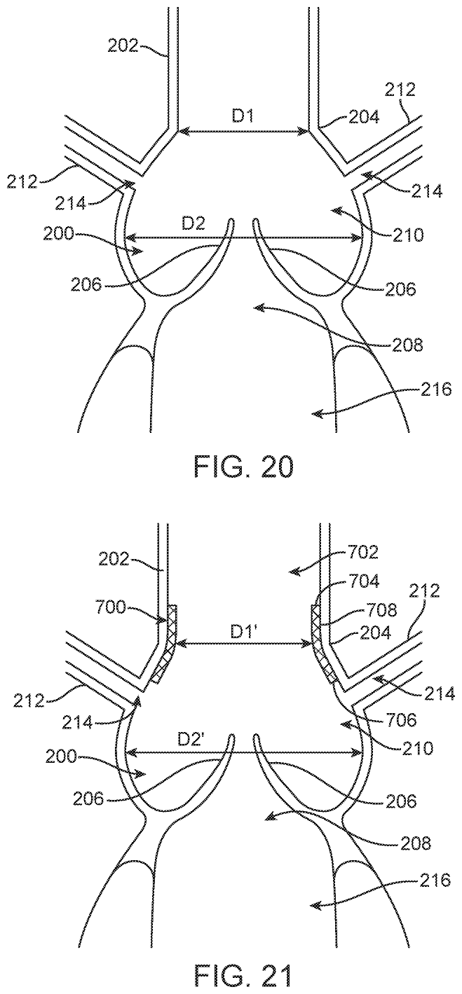

Other aspects of the present disclosure address coronary ostium access concerns raised by an implanted prosthetic aortic heart valve by deploying a stent designed to increase a diameter of various anatomy before the prosthesis is deployed. As a point of reference, FIG. 20 illustrates portions of the anatomy of and proximate the aortic heart valve 200 as mentioned above, including the aorta 202, the sinotubular junction (“STJ”) 204, the native valve leaflets 206, the aortic valve annulus 208, the sinus region 210, the coronary arteries (or “coronaries”) 212 each having a coronary ostium 214, and the left ventricle 216. Also labeled in FIG. 20 are the STJ diameter D1 and maximum sinus diameter D2. With this in mind, FIG. 21 illustrates a sinus stent 700 of the present disclosure. The sinus stent 700 defines a lumen 702, a first end 704 opposite a second end 706, and an exterior face 708. The sinus stent 700 is sized and shaped for implantation at the STJ 204, and is configured to have, in an expanded a condition, a diameter that is greater than the native, expected STJ diameter D1 (FIG. 20). When deployed, the exterior face 708 contacts the aorta 202 in a region of the STJ 204, and the sinus stent 700 exerts a radially expansive force onto the native anatomy. The sinus stent 700 widens or increases the diameter of the STJ 204 to an increased STJ diameter D1′. The so-configured sinus stent 700, would upon deployment, can also increase the diameter of the sinus region 210 to an increased maximum sinus diameter D2′ (it being understood that the increase in the STJ diameter D1 is greater than the increase in the maximum sinus diameter D2 in some embodiments).

The sinus stent 700 can be the same as or similar to a conventional stent design, having a normal, expanded condition or arrangement (shown in FIG. 20) and collapsible to a compressed condition or arrangement for loading within a delivery device, such as a catheter. In one example, the sinus stent 700 includes a plurality of struts or wire segments arranged relative to each other to provide a desired compressibility and strength to the stent frame. The struts or wire segments are arranged such that they are capable of self-transitioning from, or being forced from, a compressed or collapsed arrangement to a normal, radially expanded arrangement. The struts or wire segments can be formed from a shape memory material, such as a nickel titanium alloy (e.g., nitinol). The sinus stent 700 can be laser-cut from a single piece of material, or can be assembled from a number of discrete components. Regardless, the sinus stent 700 is configured such that when deployed at the sinotubular junction 204 in the expanded arrangement, the STJ diameter is increased at least 4 mm. In some embodiments, the STJ diameter is increased in a range between 4-8 mm. A prosthetic heart valve (not shown) can then be implanted, for example to the aortic valve annulus 208. The increased STJ diameter D1′ effected by the sinus stent 700 provides an enlarged area for improved blood flow to the coronary arteries 212, and additional space around the implanted prosthetic heart valve for accessing the coronary ostia 214. Further, the increased maximum sinus diameter D2′ effected by the sinus stent 700 can provide additional space for a guide catheter or other access device to access the coronary arteries 212.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A prosthetic support structure for supporting an aortic heart valve, the support structure being implantable in the vicinity of a native aortic heart valve and having a central passageway and an auxiliary passageway, the central passageway sized for supporting an aortic heart valve, the auxiliary passageway having an inlet and an outlet and extending along an arc of at least 20 degrees to facilitate passage of a device therethrough and toward a coronary artery when the support structure is positioned along a region of the sinotubular junction.

2. The prosthetic support structure of claim 1 in combination with a heart valve, the heart valve being positioned in the central passageway.

3. The prosthetic support structure of claim 1, wherein the prosthetic support structure comprises a first tubular frame member, a second tubular frame member disposed within the first tubular frame member, and joint members extending between and interconnecting the first and second tubular frame members.

4. A prosthetic support structure for supporting a prosthetic heart valve, the support structure being implantable in the vicinity of a heart valve and comprising a first tubular frame member and a second tubular frame member, the second tubular frame member being disposed in the first tubular frame member, the second tubular frame member having a central through passageway sized for receiving the prosthetic heart valve, the first and second tubular frame members forming an annular space therebetween, the annular space having an inflow end and an outflow end and being sized to facilitate passage of a medical device therethrough.

5. The prosthetic device support structure of claim 4, wherein the annular space is sized such that when the support structure is positioned along a region of the sinotubular junction, the medical device can be passed through the annular space and toward a coronary artery.

6. The prosthetic support structure of claim 5, further including joint members extending between and interconnecting the first and second tubular frame members.

7. A system for treating a defective heart valve, the system comprising:
a spacer assembly having a compressed configuration for delivery within a vasculature and an expanded configuration, the spacer assembly including:
an outer frame member defining a central passage, a longitudinal axis, an outflow end, and an inflow end opposite the outflow end in a direction of the longitudinal axis,
an inner frame member,
joint members extending between and interconnecting an interior face of the outer frame member and an exterior face of the inner frame member,
wherein the spacer assembly is configured such that in the expanded configuration:
at least a portion of the inner frame member is disposed within the central passage and is radially spaced from the interior face of the outer frame member by the joint members to establish a radial spacing,
at least one auxiliary passageway is defined along the radial spacing, wherein the auxiliary passageway has a central angle of at least 30 degrees relative to the longitudinal axis in continuous extension between the outflow and inflow ends, and is free of the joint members; and
a valve structure;
wherein the system is configured such that upon final implantation, the outer frame member contacts native tissue and the valve structure is supported by the inner frame member.

8. The system of claim 7, wherein the auxiliary passageway is configured for passage of an auxiliary access device.

9. The system of claim 7, wherein the system is configured such that upon final implantation, access to a native artery ostium is provided through the auxiliary passageway.

10. The system of claim 7, wherein the valve structure is permanently secured to the inner frame member.

11. The system of claim 10, wherein a longitudinal length of the inner frame member is greater than a longitudinal length of the outer frame member.

12. The system of claim 7, further comprising:
a stent;
wherein the valve structure is permanently attached to the stent.

13. The system of claim 12, wherein the valve structure and the stent are provided as components of a prosthetic heart valve having a compressed condition for delivery within a vasculature and an expanded condition.

14. The system of claim 13, wherein the system is configured such that prior to final implantation, the spacer assembly is physically separate from the prosthetic heart valve.

15. The system of claim 13, further comprising complementary mating features provided with the spacer assembly and the prosthetic heart valve configured to secure the prosthetic heart valve to the spacer assembly upon final implantation.

16. The system of claim 15, wherein the complementary mating features includes:
a protrusion extending from one of the stent and the inner frame member; and
an aperture defined by an other of the stent and the inner frame member;
wherein the aperture is sized and shaped to selectively capture the protrusion.

17. The system of claim 15, wherein the complementary mating features include a ledge defined by the inner frame member and an edge of the stent, wherein the ledge is sized and shaped to receive the edge in the expanded condition of the prosthetic heart valve.

18. The system of claim 17, wherein the ledge defines a slot, wherein the prosthetic heart valve includes a paddle, and wherein the slot is sized and shaped to selectively receive the paddle.
